(19) United States
(12) Patent Application Publication
(10) Pub. No.: US 2007/0134302 A1
(43) Pub. Date: Jun. 14, 2007

(54) Antimicrobial substrates with peroxide treatment

(76) Inventors: David W. Koenig, Menasha, WI (US); Ali Yahiaoui, Roswell, GA (US); Alison S. Bagwell, Cumming, GA (US)

Correspondence Address: KIMBERLY-CLARK WORLDWIDE, INC., 401 NORTH LAKE STREET, NEENAH, WI, 54956, US

(21) Appl. No.: 11/301,546

(22) Filed: Dec. 13, 2005

(51) Int. Cl. A61K 9/70; A61K 33/40

(52) U.S. Cl. 424/443; 424/616

(57) ABSTRACT

An oxidizing antimicrobial treatment and products containing such treatment are described. The treatment involve, in part, preparing a substrate to accept an attachment of charged moieties, and a number of stabilized peroxide compounds on at least part of a surface of said substrate. When microbes, such as bacteria, having a net charge opposite to that of the charged moieties come in close proximity to the treated substrate surface, peroxide molecules from the substrate are activated and released to kill the microbes.

### FIELD OF INVENTION

The present invention relates to products that are treated with an antimicrobial formulation that can rapidly kill a broad spectrum of microorganisms, while concurrently not introducing into the environment substances toxic to humans or other mammalian animals. In particular, the products contain a stabilized peroxide compound or mixture on at least a portion of a surface of a protective or cleaning article. When activated in the presence of moisture, the peroxide compound yields oxygen radicals that kill microbes that are near the surface of the article.

### BACKGROUND

In recent years, the prevalence of nosocomial infections has had serious implications for both patients and healthcare workers. Nosocomial infections are those that originate or occur in a hospital or long-term care, hospital-like settings. In general nosocomial infections are more serious and dangerous than external, community-acquired infections because the pathogens in hospitals are more virulent and resistant to typical antibiotics. Nosocomial infections are responsible for about 20,000-100,000 deaths in the United States per year. About 5% to 10% of American hospital patients (about 2 million per year) develop a clinically significant nosocomial infection. These hospital-acquired infections (HAIs) are usually related to a procedure or treatment used to diagnose or treat the patient's illness or injury.

The mechanism of action of nosocomial infections, as in any other infectious disease, is dependent on host, agent and environment factors. Risk factors for the host are age, nutritional status and co-existing disorders. Nosocomial infections are influenced by the microbes' intrinsic virulence as well as its ability to colonize and survive within institutions. Diagnostic procedures, medical devices, medical and surgical treatment are risk factors in the hospital environment. Hospital-acquired infections can be caused by bacteria, viruses, fungi, or parasites. These microorganisms may already be present in the patient's body or may come from the environment, contaminated hospital equipment, healthcare workers, or other patients. Depending on the causal agents involved, an infection may start in any part of the body. A localized infection is limited to a specific part of the body and has local symptoms.

In today's healthcare environment, the battle against nosocomial infections has not yet been won. Even though hospital infection control programs and a more conscientious effort on the part of healthcare workers to take proper precautions when caring for patients can prevent about 25% to 33% of these infections, a significant number of infections still occur. The current procedures are not sufficient. Despite enforcement of precautionary measures (e.g. washing hands, wearing gloves, face mask and cover gowns), HAIs still occur predominately via contact transfer. That is, individuals who contact pathogen-contaminated surface such as hands, clothing and/or medical instruments, can still transfer the pathogens from one surface to another immediately or within a short time after initial contact. Researchers have employed numerous ways to attack microbe related issues. Antiseptics and disinfectants are used extensively in hospitals and other health care settings for a variety of topical and hard-surface applications. In particular, they are an essential part of infection control practices and aid in the prevention of nosocomial infections. Conventional antimicrobial agents currently available, however, are not very effective at killing and immobilizing pathogens on to the surfaces to which the antimicrobial agents are applied.

The problem of antimicrobial resistance to biocides has made control of unwanted bacteria and fungi complex. The widespread use of antiseptic and disinfectant products has prompted concerns about the development of microbial resistance, in particular cross-resistance to antibiotics. A wide variety of active chemical agents (or “biocides”) are found in these products, many of which have been used for hundreds of years for antisepsis, disinfection, and preservation. Despite this, less is known about the mode of action of these active agents than about antibiotics. In general, biocides have a broader spectrum of activity than antibiotics, and, while antibiotics tend to have specific intracellular targets, biocides may have multiple targets. The widespread use of antiseptic and disinfectant products has prompted some speculation on the development of microbial resistance, in particular cross-resistance to antibiotics. This review considers what is known about the mode of action of, and mechanisms of microbial resistance to, antiseptics and disinfectants and attempts, wherever possible, to relate current knowledge to the clinical environment.

Antibiotics should only be used when necessary. Use of antibiotics creates favorable conditions for infection with the fungal organism Candida. Overuse of antibiotics is also responsible for the development of bacteria that are resistant to antibiotics. Furthermore, overuse and leaching of antimicrobial agents or antibiotics can cause bioaccumulation in living organisms and may also be cytotoxic to mammalian cells.

To better protect both patients and healthcare providers, protective articles, such as garments, gloves, and other coverings that have fast-acting, highly efficient, antimicrobial properties, including antiviral properties, are need for a variety of different applications for wide spectrum antimicrobial protection. The industry needs anti-microbial materials that can control or prevent contact transfer of pathogens from area to area and from patient to patient. In view of the resistance problems that may arise with conventional antimicrobial agents that kill when bacteria ingest antibiotics, an antimicrobial that kills virtually on contact and has minimal or no harmful byproducts or residue afterward would be well appreciated by workers in the field. Hence, it is important to develop materials that do not provide a medium for the pathogens to even intermittently survive or grow upon, and that are stably associated to the substrate surfaces on which the antimicrobial agent is applied. Moreover, the antimicrobial protective articles should be relatively inexpensive to manufacture.

### SUMMARY

The present invention pertains to a protective or cleaning article that has an exterior surface with at least a partial coating or layer of a stabilized peroxide compound associated with the exterior surface, which can be used for antimicrobial uses. The protective or cleaning article can be made from a variety of polymer-based materials, depending on the particular configuration and use of the article. For instance, the article can have a substrate that is composed in part from a natural or synthetic polymer latex film, natural cellulose fibers or weave, or a flexible non-woven web (e.g., spunbond, meltblown, or laminate combinations thereof (e.g., SMS)). Both the latex film and non-woven web can be elastomeric. The non-woven web can have either machine-direction (MD) or cross-directionally (CD) elastic characteristics. In the realm of medical or infection-control uses, for example, latex films are typically part of protective articles such as gloves, and non-woven webs are used in face masks and cover gown. In household or cleaning applications, elastomeric latex films and non-woven materials can be fashioned into a number of products. For instance, cleaning wipes take up and trap dirt, or gloves protect a user's hands from contacting or transferring the dirt. The presence of a peroxide releasing compound on the surface of such article can greatly enhance their cleaning and antimicrobial benefits.

### BRIEF DESCRIPTION OF FIGURES

FIG. 1 is a series of schematic diagrams illustrating the antimicrobial mechanism of the present invention.

FIG. 2 is a series of schematic representations illustrating the interaction between a microbe and a substrate surface.

FIG. 3 shows a glove that has been prepared with an antimicrobial treatment according to an embodiment of the present invention.

### DETAILED DESCRIPTION OF THE INVENTION

### Section A

The antimicrobial efficacy and potency of biocides are highly dependent on several chemical, physical, and environmental factors. Among these factors, the more important ones include the formulation and concentration of active agents, temperature, pH, duration of exposure, the physiological state and population size of the target microbes, and the presence of ions and organic matter. Also, the physical and chemical characteristics of the substrate to be disinfected can be important because of the interaction that the substrate may have with the biocide.

The inactivation or killing of microorganisms by means of either controlling their reproductive or metabolic activities typically is not an instantaneous event. In most situations, the greater the concentration of a particular antimicrobial agent, the faster the rate of microorganism inactivity, or the longer duration of exposure of a microbe to a disinfectant or biocide, the greater the antimicrobial effectiveness increases.

In recent years, a fast-acting antimicrobial treatment that is non-leaching from products or substrate surfaces has been in demand. The active agent of the antimicrobial treatment should not be either harmful to human skin or result in a toxic residue, which may breed resistant microbial strains. The active agents of the antimicrobial composition, if released into the immediate microenvironment decompose into benign components, predominantly oxygen and water, which are non-toxic to human skin or mammalian physiological systems.

At present, biocides can be categorized into four classes. They include: 1) toxic organic chemicals, 2) surfactant-based compounds, 3) metal or metallic molecules, and 4) oxidizing antimicrobial agents. Toxic organic chemicals that include, for example, thiazoles, thiocynates, isothiazolins, cyanobutane, dithiacarbamates, thione, triclosans, and bromo-compounds, while effective, have a residual toxicity in the local environment than can be harmful to the human user. Likewise, metal compounds are usually slow acting, environmentally persistent and toxic. Surfactants can be disrupt bacterial cell membranes, but they are also relatively-slow acting, not always broad spectrum, and persistent. On the other hand, oxidizing compounds have a broad spectrum and kill microbes rapidly. A shortcoming of conventional oxidizing preparations is that they are relatively short duration. The oxidizing antimicrobial agents include such compounds as halogens, halogen-containing polymers, chlorine dioxide, hydrogen peroxide, and ozone, which are relatively fast-acting and having a broad biocide spectrum.

The present invention describes a substrate that has a charged surface to readily attract oppositely charged microbes, such as bacteria, fungi and viruses, and at least a partial coating or layer of a stabilized peroxide compound. For examples, cationic molecules will attract and bind negatively charged microbes. Also disposed on the substrate surface is a plurality of stabilized oxidizing compounds. When activated in the presence of free moisture, such as liquid water or water vapor, the oxidizing compound releases from the surface. As one of the best kinds of biocides, oxidizing compounds provide effective quick-kill and broad-spectrum action, with minimal potential to develop antibacterial resistance. Oxidizing compounds such as hydrogen peroxides have been used for cleaning wounds or surgical sites after closure. The activity of peroxides is greatest against anaerobic bacteria. Furthermore, hydrogen peroxide has virucidal properties.

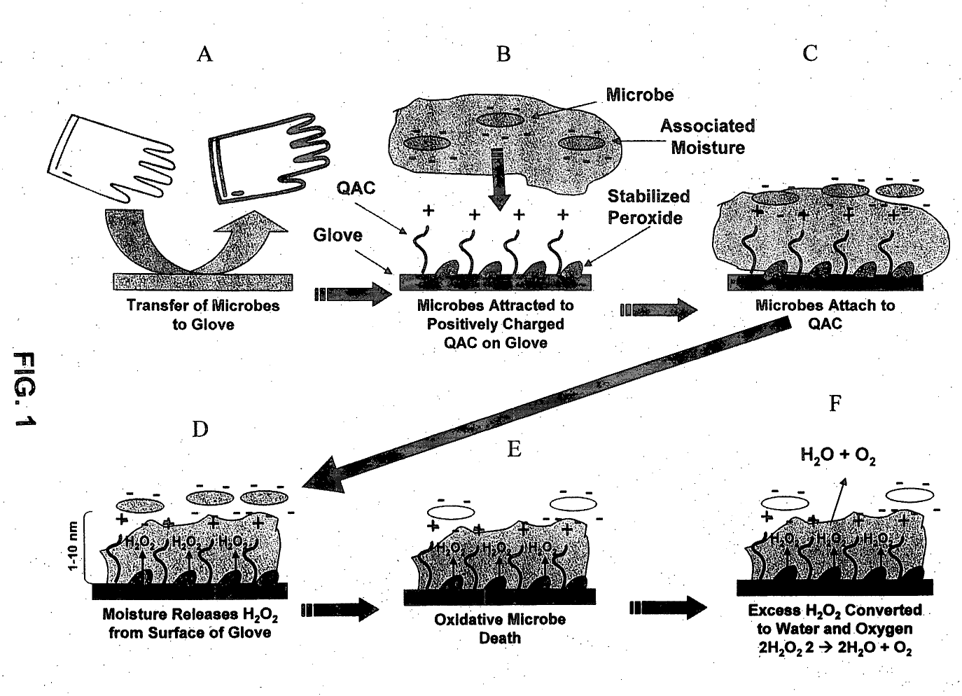

The present invention provides a simple and elegant mechanism for addressing the build up of often toxic agents on treated surfaces. FIG. 1, depicts in a series of schematic diagrams one way the present invention kills adsorbed microbes. In the embodiment, FIG. 1A shows a glove coming in contact with a contaminated surface or skin, and transferring the microbial contaminants to the surface of the glove. FIG. 1B is a magnified view at the surface of the glove as microbes come into contact with the glove substrate. Microbes typically exist in environments that allow for a micro-envelope of moisture surrounding their cells. According to the embodiment shown, negatively charged microbes are attracted to cationic moieties on the surface of the glove. In other embodiments, negatively charged surface moieties can be adapted to draw in positively charged microbes. A number of stabilized peroxide molecules are situated on the surface of the glove substrate. When the microbes attach to the cationic moieties, the micro-envelope of moisture around the microbes also draws near and interacts with the glove surface, activating and releasing peroxide from the surface, as illustrated in FIGS. 1C and 1D. The oxidative effect of the peroxide release kills the microbes that have become attached to the substrate in FIG. 1E. Excess hydrogen peroxide generated by the system, instead of becoming a problem, will decompose to harmless water and molecular oxygen and dissipates from the microenvironment of the substrate as illustrated in FIG. 1F.

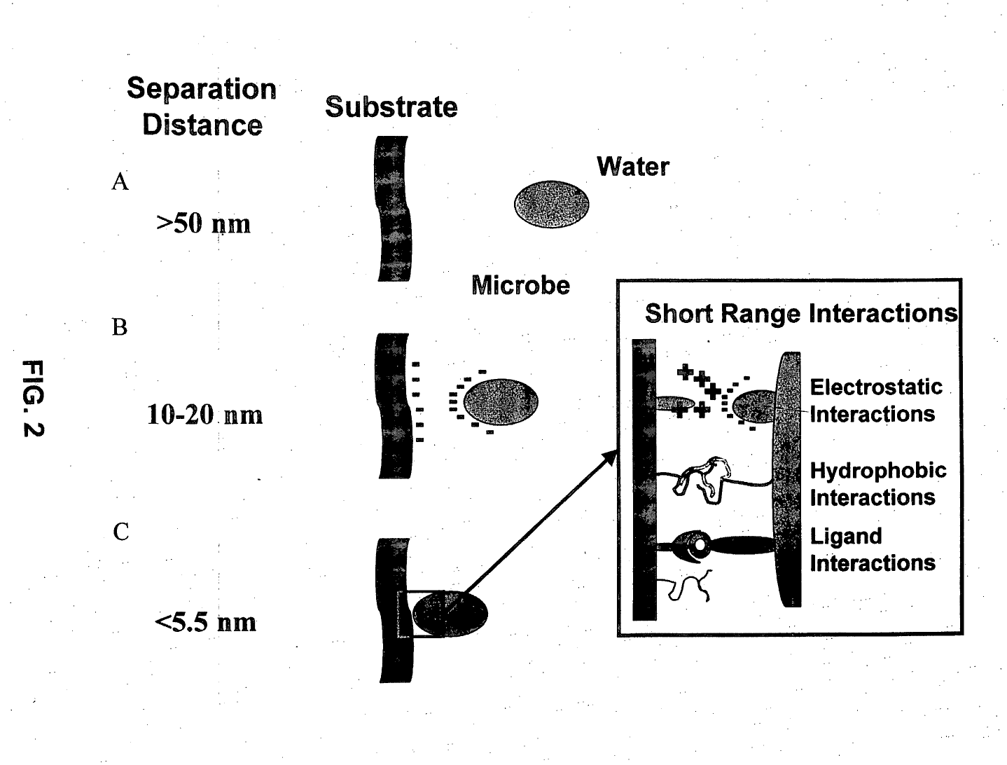

FIG. 2, shows a series of schematic panels illustrating the interaction of a microbe with a substrate surface. The microbe can be present either in a liquid medium, such as water, or have a moisture or biological envelope around its outer surface or cellular membrane. The diagram shows the relative distances between the microbe and the substrate surface and the different physical or chemical events as the microbe approaches the substrate. In the top panel, the microbe is greater than 50 nm away from the substrate; there is minimal interaction between the two. As the microbe approaches to within about 25 nm, electrostatic charge interactions between the substrate and microbe begin to appear. At relatively close distances of less than about 10 nm or 5 nm from the substrate, three kinds of significant surface to microbe interactions either strength or begin to occur. These typically involve: electrostatic, hydrophobic, or ligand interactions. (See, Habash, M. and G. Reid, Microbial Biofilms: Their Development and Significance for Medical Device-Related Infections, J. Clinical Pharmacology 39:887-898, 1999.) When in close proximity to the surface, the effective peroxide-release atmosphere about the coated substrate surface is within about 100 nm of the surface, more typically within 50 nm. Desirably, the peroxide micro-atmosphere is operational within about 20-25 nm, and optimal within about 5-10 nm of the surface.

Most biological entities have a net negative charge, positively charged membrane organisms will want to go to the membrane, targeted concentration. Charges moieties such as cationic compounds impart a charge to the substrate surface to attract charged microbes into close proximity with the peroxide prepared substrate surface. The cationic compounds contained in the products of the present invention appear to electrostatically interact with contaminants and other soils and inorganic particles, which contact the surface of the protective article and binds the contaminant such that it may be secured away from a user's skin. As used herein, the term “contaminant” should be read to include Gram negative and Gram positive bacteria, fungi and fungal spores, yeasts, molds and mold spores, protozoan, and viruses.

Hydrogen peroxide is a broad spectrum oxidizing agent, and is often used to clean wounds. When peroxide is released in sufficient quantities in a microenvironment, such as against potentially harmful organic compound molecules or microorganisms, peroxide will oxidize the compounds and/or surface lipids, proteins, or carbohydrates. Typically, since cellular membranes or viral caspids contain at least one of these three components, extreme oxidization will overwhelm the natural ability of microorganisms to cope with oxidation, and either denature the cellular membrane, rendering cellular metabolic reactions inoperative, or rupture the virus, releasing its genetic material and killing the organism. The resulting molecular oxygen and water vapor are benign by products that overcome the problem of persisting toxins in the environment. The activity of peroxides is greatest against anaerobic bacteria.

Stabilized peroxides have been blended in solutions with iodophores or quaternary ammonium compound, which have been used for disinfection of equipment surfaces. Stabilized peroxides are effective against a broad range of pathogens, such as both enveloped and non-enveloped viruses, vegetative bacteria, fungi, and bacterial spores. Similar to formulations found in peroxide-containing tooth gel or paste, the peroxide containing salts or compounds can be mixed with stabilizers that prevent the peroxide from releasing prematurely. It is desired that only in the presence of a sufficient amount of moisture will the peroxide react.

The hydrogen peroxide sources can be selected from a group including perborate compounds, percarbonate compounds, perphosphate compounds, and/or mixtures thereof. According to an embodiment, the stabilized peroxide-containing compound can be in the form of a carbohydrate mixture or salt. For example, as described in detail in U.S. Pat. No. 6,887,496, the contents of which are incorporated herein, the oxygen producing compounds for incorporation may include, for example, a carbohydrate-hydrogen peroxide mixture which has been crystallized into a stable crystalline material. Preferably, the oxygen producing compound is a crystalline compound comprised of a sugar alcohol-hydrogen peroxide mixture, such as mannitol-hydrogen peroxide or sorbitol-hydrogen peroxide. Polysaccharides such as cyclodextrin serve as carriers for organic peroxides. Guest-host complexes in which the cyclodextrin hosts stability holds the guest peroxide molecule or compounds; in particular, organic type of peroxides typically have a hydrophobic moiety that is situated in the cavity of the host while the peroxide moiety extend outside to react with the microbes.

The attractive forces, such as electrostatic, hydrogen bonding, polar, apolar, or van der waals forces, between the peroxide and the carrier molecules can be tailored to control the kinetics of peroxide release or interaction with the environment. Alternatively one can design the carrier to regulate the extent or level of exposure that the peroxide moieties have with the outside environment. A carrier, such as cyclodextrin, can encapsulate in-part or fully the peroxide moieties. Alternatively, one can use ligand or chelation mechanisms to regulate the exposure of the peroxide moiety to environmental hydrogen or organic molecules that may trigger the release of active peroxide.

Water soluble polymers can be employed as carrier for the peroxide salts. Some other materials that can be used to make the peroxide compound may be applied as a salt, and may include, for example, urea peroxide or urea hydrogen peroxide (CH₄N₂O.7H₂O₂) (Also referred to as carbamide peroxide. See, “Regulatory and Ingredient Use Information,” regarding the labeling names for U.S. OTC Drug Ingredients in Volume 1, Introduction, Part A.), employed in stabilized amides (including salts; excluding alkanolamides and alkoxylated amides); sodium carbonate peroxide (CH₂O₃. 3/2H₂O₂.2Na) (peroxy-sodium carbonate or sodium percarbonate); calcium peroxide (CaO₂) oxidizing agent; PVP-hydrogen peroxide, a complex of polyvinylpyrrolidone and hydrogen peroxide ((C₆H₉NO)ₓ.½H₂O₂); or 2-pyrrolidinone, 1-ethenyl-, homopolymer, compounded with hydrogen peroxide (H₂O₂) (2:1). Ethyl-hydroxyethyl cellulose can be a carrier for hydrogen peroxide or other peroxides.

It is envisioned that certain stabilizer components can be incorporated to prevent a mass activation and release of peroxide when the coated substrate is exposed to an aqueous environment or other liquids. For instance, a stabilizer or carrier molecule can be covalently attached to the substrate by means of radiation grafting and load the peroxide moieties onto the covalently attached carriers. A radiation-induced graft polymerization of a hydrophilic monomer onto a substrate can take the form of a hydrogel graft, according to a method such as described in U.S. Pat. No. 6,387,379, incorporated herein, which can act as a host for a peroxide compound, thus forming a hydrogel-peroxide complex. A hydrogel is a hydrophilic polymer that can be crosslinked to form a cohesive network so that it swells in water but does not necessarily readily dissolve in water. For instance, a hydrophilic monomer such as N-vinyl pyrrolidone (NVP) can be used. Other hydrophilic monomers listed in U.S. Pat. No. 6,387,379 can also be used. As for radiation sources, ultraviolet (UV), gamma ray, or electron beam can be used.

An example of a formulation (Table 1) would contain a mixture of quaternary ammonium compounds (QACs) and stabilized peroxide, such as urea peroxide, calcium peroxide, sodium carbonate peroxide, mannitol and/or sorbitol peroxide. Urea peroxide, also known as carbamide peroxide, is a common ingredient in tooth paste and other dental bleeching systems. A formulation containing about 10% carbamide peroxide exhibits a similar level of active agent as another formulation containing about 3.3% hydrogen peroxide. The amount of stabilized peroxide present on the treated substrate can be up to about 20 percent by weight, but more typically is present at about 10-12 or 15 percent by weight. Desirably, the amount of active peroxide on the surface can be about up to about 7 or 8 percent, and preferably about up to about 4 or 5 percent.

TABLE 1
Formulation

| Ingredient (Wt. %) | Formula 1 | Formula 2 |
|---|---|---|
| Stabilized peroxide | 1-20% | 1-15% |
| Quaternary Ammonium Compound (QAC) | 4.0% | 2% |
| Cetyl pyrridinium chloride | 0.1% | 0.1% |
| Q25211 wetting agent | 0.01% | 0.01% |
| Anti-forming agent | 0.002% | 0.002% |
| Deionized water | QS | QS |

A complex carbohydrate-hydrogen peroxide mixture, according to an embodiment, is introduced into or onto a substrate in an amount sufficient to produce a stream of oxygen upon insult such that it hinders the metabolism of microbes on and near the surface of the treated substrate. The mixture is capable of generating oxygen upon activation, and the oxygen acts as a terminal electron acceptor for bacteria on or near the substrate surface, such that the bacteria is either killed or the production of toxic or volatile organic compounds by bacterial is reduced or neutralized.

A fast-acting oxidizing microenvironment is neutral or benign to humans, mammals, or other macroorganism, but can be deadly to most microorganisms. A concentrated release of peroxide can overpower a microbe's normal ability to use catalyase—an enzyme that degrades hydrogen peroxide—and protect itself from oxidizing agents. The rapid and overwhelming action of reactive oxygens oxidizes and decomposes any exposed organic structures, including lipids, lipid membranes, and membrane proteins, beyond the ability or capacity for the cell to repair itself. Hence, the microbial cell dies. Even a viral protein coat of a virus can be irrevocably damaged by rapid oxidation resulting in either the molecular inactivation or death of the virus.

The present peroxide coating can produce a broad spectrum, quick kill of about 90% of bacteria in a given sample within about 15 minutes by oxidizing or dissolving all organic matter for no recoverable bacteria population. Preferably, the oxidization exhibits a 95% or better microbe kill rate within about 10 minutes, and more preferably about a 95% rate at about 5 minutes or less after contact.

The formulations can be applied to the substrate or incorporated within the substrate surface. The peroxide compound can be applied to either polymer-based elastomeric or non-woven materials through a variety of processes, such as heated spray coating, dip and squeeze in a bath or spaying, or Gravier or Meyer rod processes can be used to add the formulation to the substrate surface with air drying. Preferably the substrate is coated with an evenly distributed, uniform layer of the antimicrobial cationic and stabilized peroxide compounds. The substrate may be made from a variety of materials, including for example, elastic polymers, olefins, natural and synthetic fiber-based sheets and laminates, and may take the form of a membrane, or geometric solid.

To ensure that the peroxide compounds are not activated prematurely, a number of treated protective or cleaning articles can be stored in an air-tight, dry container, such as bags or jars, preferred, with a desiccating packet to maintain low moisture content with the container.

It is envisioned that the peroxide containing coating can be applied to a number of articles that can be found in hospital/health care, food preparation, industrial, institutional, or home settings. These articles, may include gloves, cover gowns, or cleaning substrates or wipers, but probably not suited for skin-contacting surfaces or materials in absorbent personal care products, such as diapers.

Currently, gloves have been developed to limit the transfer of microbes from the glove to environmental surfaces. This technology employs a coating of quaternary ammonium compounds (QAC) on the external surfaces of the glove substrate, which serves as an attractant of microbes through an electrostatic charged mechanism. This mode of action uses the net negative charge associated with the surfaces of biological or microbial cells, which are attracted to the cationic charge of QAC on the substrate. This technique has been effective to increase the removal of microbes from skin when using wipes and other articles that have been impregnated with cationic compounds.

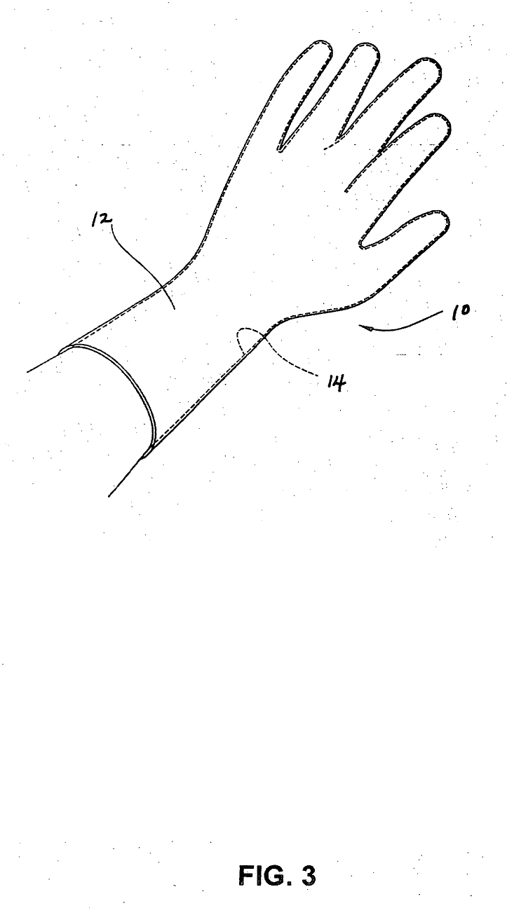

In the healthcare and hospital environment, contamination or improper handling of many materials, instruments, and other articles that may contact patients can be a route of infection transfer. The ability to impart a rapid acting antimicrobial agent or coating to natural and synthetic polymer latex gloves would be a significant improvement in controlling cross-contamination between clinician and patient. According to embodiments, such as examination or work gloves or other garment articles that are worn against or in close proximity to human skin, the peroxide enabled surface is typically applied to the final outer surface, directed away from the wear's skin. FIG. 3, is a general representation of a glove 10 with a surface 12 that can be treated with stabilized peroxide compounds 14, which when activated can generate an oxidizing micro-atmosphere near or around the surface of the glove to kill microbes that are near of in contact with the surface.

In embodiments that use a carbohydrate-hydrogen peroxide or a hydrogel-hydrogen peroxide mixtures for reducing the amount of microbes, the process for preparing a product involves mixing a carbohydrate or a hydrogel and hydrogen peroxide and then freeze drying the mixtures to remove any solvent in the mixture and produce solid particles. (See for example, detailed description in “A Guide to Freeze Drying for the Laboratory,” LABCONCO, Kansas City, Mo., 2004, (www.labconco.com).) Because certain peroxides are typically sensitive to heat, which may deactivate the compound, a freeze-drying process is desirable. The temperature of the mixture in solution is lowered (generally about −25° C. or −30° C.) to well below the freezing temperature of water and the water is sublimated off.

Alternatively, some other peroxide compounds can be prepared according to a hot or heated approach to drive off water in making the peroxide compound, such as an alcohol hydrogen peroxide mix (e.g., mannitol peroxide combination). This process can stabilize the sugar and alcohol mixture. The mixture is heated to a temperature of at least about 90° C. for at least about 4.5 hours to evaporate water. Desirably, the mixture is heated to a temperature of about 97° C. for about 7 hours. Finally, the solid particles produced are incorporated into the product. In certain iterations, the material is heated at a higher temperature at about 100-110° C. for up to 4.5 hours. (See further, S. Tanatar, “Double Compounds of Hydrogen Peroxide with Organic Substances,” JOURNAL OF THE RUSSIAN PHYSICAL CHEMICAL SOCIETY, 1909, 40:376.)

The freeze-drying process, however, is likely to provide a higher yield end product then a heating method depending on the kind of peroxide product desired.

For urea-peroxide compositions, there is no need for heating step. A dry sample of the peroxide compound should have less than about 2-5% hydration content by weight. The dry peroxide compound can be milled into a powder with a mean particle size of about 5 nm or smaller. Agglomerations of the peroxide particles can be are about 15-20 nm or smaller.

A process for treating a substrate with an oxidizing compound, the process may involve providing a peroxide-containing compound and applying it either onto a surface of the substrate of the substrate or incorporating it into the substrate such that the peroxide-containing compound is generated in-situ on or in the substrate, provided that sufficient moisture is able to permeate into the substrate to interact and activate with the peroxide compound. The in-situ formation of peroxide can be accomplished by means of either a freeze-dry method or a heated method, such as described above. The predetermined choice of method can depend on the type or nature of the peroxide-containing compound and/or the physical properties or characteristics of the substrate.

During the application of peroxide it is desirable to minimize exposing the treated substrate to heat so that the peroxide moieties are not prematurely deactivated or reacted with the immediate environment. One can apply a first layer or coating that includes a carrier or host for the peroxide. This coating may also contain another class or type of antimicrobial agent. Following drying of the first layer a peroxide formulation is applied onto the first layer to associate with the carriers with minimal drying. This application can be done by means of a variety of techniques, including spray coating or roller applicators. In another embodiment, the second peroxide layer can be an anhydrous, powder such as CaO₂ or a non-aqueous organic peroxide, without need for drying. In another example, after applying the first layer, one can also use a printing process, such as, valve-jet, digital, or piezoelectro devices, to apply micro-droplets of peroxide solution in localized areas or patterns in similar fashion as inks for creating in printed graphics.

The peroxide compounds can be associated directly with or on the treated substrate surface. Alternatively, it is envisioned in certain embodiments that a product according to the invention may have as part of the exterior or active surface of a substrate degradable hollow structures, such as fibers, filaments, beads or other forms, in which one can fill and store peroxide agents. A source of significant moisture or the presence of specific biological or microbial secretions may serve as a trigger to breakdown the hollow structure. Once the substrate contacts such triggers, the encapsulating hollow structures may begin to dissolve and release the peroxide within, in either a prolonged, measured fashion or fast, explosive fashion onto the substrate surface to kill against nearby microbes.

### Section B

A variety of different kinds of substrates can be treated or coated with the present antimicrobial composition. According to certain embodiments, the substrate materials may include, for example, elastomeric membranes, films or foams, such as natural rubber or synthetic polymer latex, soft and hard rubber or plastics, or metal, glass or ceramic surfaces, such as found with medical devices and/or surgical equipment and instruments, or hospital physical plant. Alternatively, other embodiments may have substrate materials that are selected from either woven or non-woven fabrics. Woven fabrics may be made from natural fibers (e.g., cellulose, cotton, flax linen) or a blend of natural and synthetic fibers (e.g., thermoplastics, polyolefin, polyester, nylon, aramide, polyacrylic materials). A wide variety of elastic or non-elastic thermoplastic polymers may be used to construct non-woven substrate materials. For example, without limitation, polyamides, polyesters, polypropylene, polyethylene, copolymers of ethylene and propylene, polylactic acid and polyglycolic acid polymers and copolymers thereof, polybutylene, styrenic co-block polymers, metallocene-catalyzed polyolefins, preferably with a density of less than 0.9 gram/cm³, and other kinds of polyolefins, for the production of various types of elastic or non-elastic fibers, filaments, films or sheets, or combinations and laminates thereof.

A nonwoven web or laminate can be treated with compositions and methods of the present invention to impart broad spectrum anti-microbial and antistatic properties at desired or predetermined locations on the substrate, while maintaining desired physical or mechanical properties. Furthermore, the components of the treatment composition can be applied in separate steps or in one combined step. It should also be understood that the method and anti-microbial surface treatment of nonwoven materials with topical application of ingredients of this invention may incorporate not only multiple ingredients for improved anti-microbial performance but may also be used to incorporate other ingredients, such as anti-static agents which may afford dissipation of static charge build up, and skin care agents such as emollients.

Embodiments of the present antimicrobial composition may include a protective article, such as gloves, face masks, surgical or medical gowns, drapes, shoe covers, or fenestration covers. For purpose of illustration, the beneficial properties of the present invention can be embodied in a facemask containing a combination of one or more antimicrobial agents and co-active agents that rapidly inhibit and control the growth of a broad spectrum of microorganisms on the surface of the product both in the presence and absence of soil loading. The antimicrobial coating, which rapidly kills or inhibits, can be selectively placed on the exterior nonwoven facing of the mask rather than throughout the entire product. The antimicrobial agents are non-leaching from the surface of the mask in the presence of fluids, and/or are not recoverable on particles that may be shed by the mask in use and potentially inhaled by the user as measured using a blow-through test protocol. Exemplary face masks and features incorporated into face masks are described and shown, for example, in the following U.S. Pat. Nos. 4,802,473; 4,969,457; 5,322,061; 5,383,450; 5,553,608; 5,020,533; and 5,813,398. The entire contents of these patents are incorporated by reference herein in their entirety for all purposes.

The antimicrobial compositions can be applied topically to the external surfaces of nonwoven web filaments or fibers after they are formed. Desirably, a uniform coating is applied over the substrate surfaces. A uniform coating refers to a layer of antimicrobial agents that does not aggregate only at selected sites on a substrate surface, but has a relatively homogeneous or even distribution over the treated substrate surface.

Nonwoven fabrics that are treated with an antimicrobial coating of the present invention can be fabricated according to a number of processes. In an illustrative example, a method for preparing an anti-microbial treated substrate involves providing a polymer substrate and applying to the substrate the stabilized peroxide molecules. According to an embodiment, the antimicrobial composition can be applied to the material substrate via conventional saturation processes such as a so-called “dip and squeeze” or “padding” technique. The “dip and squeeze” or “padding” process can coat both sides of and/or through the bulk of the substrate with the antimicrobial composition.

The present inventive products comprise a substrate carrying a cationic compound that is highly effective in binding numerous contaminants including fungi, yeasts, molds, protozoan, viruses, soils, and other substances. Microbes are immobilized through electrostatic interactions against the cationic charged substrate. The cationic compounds impregnated into or onto the products of the present invention do not necessarily kill or inhibit the growth of microbes, but displace and bind the predominantly negatively charged microbes or other contaminants from the wound surface through positive-negative or negative-positive electrostatic interactions. This is highly advantageous in that the products of the present invention do not require an antimicrobial, bactericidal or bacteriostatic ingredient to be highly effective in safely cleaning skin. When the products of the present invention are utilized in or around skin wounds, microbes are not simply killed and left in the wound, but are actually bound to the cationic compounds in or on the fibers of the product and removed from the skin. This may significantly reduce the chance of further infection in and around the wound. Further, the cationic compounds used in the products of the present invention are substantially non-toxic and non-irritating to the wound and surrounding skin.

Without being bound to a particular theory, it appears that by increasing the attractive forces between the product containing the cationic compounds and the microbe and/or contaminant on or near the skin or wound surface in excess of the forces attracting the microbe and/or contaminant to the skin, cleaning of the skin can be significantly enhanced by dislodging and binding the contaminant to the cationic species added to the product. It appears that the cationic compounds interact with the overall net negative charge of the microbe and/or contaminant causing the detachment of the microbe and/or contaminant from the skin through an electrostatic interaction. The interaction between the cationic compounds and the microbe and/or contaminant appears to be stronger than the combined forces of adhesion that retain the microbe and/or contaminant on or near the skin including hydrophobic interactions, electrostatic interactions, and ligand interactions. Because the microbe and/or contaminant is released from the skin and bound to the charge modified product, it may be easily and efficiently carried away by the product. This is highly advantageous over more traditional products as the contaminant is not merely dislodged from the skin or wound surface, but is dislodged and then removed from the surface through interactions with the substrate containing the cationic compounds. A suitable amount of cationic compounds are added to the products of the present invention such that the forces binding the contaminant to the skin surface, such as hydrophobic interactions, electrostatic interactions, and ligand interactions, can be overcome by the attraction to the cationic species.

In accordance with the present invention, numerous microbes and soils such as, for example, Candida albicans, can be effectively captured and removed away from mammalian skin or a substrate surface by means of a cleansing product or substrate having a sufficient amount of cationic compounds, such as, for example, octadecyl-dimethyl-trimethoxyl-silpropyl-ammonium chloride, having a suitable effective charge density or anion exchange capacity which modifies the overall charge density of the product. It has been discovered that by providing a substrate comprising a sufficient amount of cationic compounds having an effective charge density of from about 0.1 microequivalents/g to about 8000 microequivalents/g or more, the substrate surface can be electrically altered such that the resulting product has a Positive Charge Index as defined herein of at least about 35 positive charge units, more typically about 50 or above, and preferably about 52-250 or 300. Such a Positive Charge Index allows numerous types of microbes and contaminants to be electrostatically dislodged from the skin surface, captured and carried away. The cationic compound-containing products of the present invention are safe for use on the skin and in and around wounds, as microbes are removed from the wound surface without a substantial risk of rupturing, and thus the risk of introduction of byproducts from the microbe into wounds is minimized or eliminated. In some desired embodiments, the substrate carries a cationic compound capable of binding contaminants located on the skin. Preferably, the cationic compound has an effective charge density of from about 500 or 1000 microequivalents/g to about 8000 microequivalents/g and the product has a Positive Charge Index of at least 52. The substrate can be made into a product comprising either a woven or a non-woven web material and a cationic compound capable of binding contaminants located on the surface of skin.

The cationic compounds described herein can be incorporated into or onto a substrate or product utilizing numerous methods. In one embodiment of the present invention, the cationic compounds are impregnated into the fibers comprising the underlying substrate of the cleansing product during the substrate manufacturing process. Although generally referred to herein as “pulp fibers” or “cellulose fibers,” it should be recognized that various types of fibers, including wood pulp fibers and synthetic and polymer-type fibers, are suitable for substrate use in the cleansing products of the present invention, and are within the scope of the present invention. Suitable substrates for incorporation of the cationic compounds include, for example, cellulosic materials, coform materials, woven webs, non-woven webs, spunbonded fabrics, meltblown fabrics, knit fabrics, wet laid fabrics, needle punched webs, or combinations thereof.

Examples of suitable cationic compounds that can be utilized to increase the overall effective cationic charge density of the cleansing products of the present invention include, for example, polyquaternary ammonium compounds, such as those sold under the tradename Bufloc 535 (Buckman Laboratories International, Memphis, Tenn.), Nalco 7607 (ONDEO NALCO Company, Naperville, Ill.), Reten 201 (Hercules Inc., Wilmington Del.), Cypro 515 (CIBA Speciality Chemicals, Suffolk, Va.), Bufloc 5554 (Buckman Laboratories International, Memphis, Tenn.), and Busperse 5030 (Buckman Laboratories International, Memphis, Tenn.) and cationic polymers, inorganic cationic species, biological cationic polymers, modified chitosan, octadecyldimethyltrimethoxylsilpropylammonium chloride, octadecyldimethoxylsilpropylammonium chloride, polyacrylamides, diallydimethylammonium chloride, dicyandiamideformaldehyde, epichlorohydrinamine, cationic liposomes, modified starch, 1-methyl-2-Noroleyl-3-oleyl-amidoethyl imidazoline methylsulfate, 1-ethyl-2-Noroleyl-3-oleyl-amidoethyl imidazoline ethylsulfate, trimethylsilylmodimethicone, amodimethicone, polyquaternium-2, polyquaternium-4, polyquaternium-5, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-32, polyquaternium-33, polyquaternium-34, polyquaternium-35, polyquaternium-36, polyquaternium-37, polyquaternium-39, polysilicone-1, polysilicone-2, and mixtures and combinations thereof. Especially preferred compounds include quaternary compounds, polyelectrolytes, octadecyldimethoxylsilpropylammonium chloride, 1-methyl-2-Noroleyl-3-oleyl-amidoethyl imidazoline methylsulfate, and 1-ethyl-2-Noroleyl-3-oleyl-amidoethyl imidazoline ethylsulfate. It would be recognized by one skilled in the art that other cationic compounds commonly used in pulp manufacturing processes could also be utilized in accordance with the present invention to significantly increase the overall cationic effective charge density of the resulting product.

The cationic compounds for incorporation into products of the present invention have a net cationic charge, and may sometimes be referred to as anion exchangers. Typically, the products of the present invention contain cationic compounds having sufficient positive charge to impart improved cleaning characteristics into the products through electrostatic interactions with microbes and/or contaminants and skin. The amount of “cationic charge” on a particular compound can vary substantially and can be measured utilizing several different units. Anionic exchangers are sometimes referred to as having a “capacity” which may be measured in microequivalents per gram or milliequivalents per gram, or may be measured in terms of the amount of a certain compound or protein that the anionic exchanger will bind. Still another way of referring to the amount of positive charge is in terms of micro or milliequivalents per unit area. One skilled in the art will recognize that the exchange capacity units can be converted from one form to another to calculate proper amounts of anion exchanger for use in the present invention.

In accordance with the present invention, the chemical additives utilized to increase the overall effective cationic charge density of the resulting product have a cationic charge. Cationic compounds useful in the present invention typically have an effective charge density of from about 0.1 microequivalents/g to about 8000 microequivalents/g, more preferably from about 100 microequivalents/g to about 8000 microequivalents/g, still more preferably from about 500 microequivalents/g to about 8000 microequivalents/g, and most preferably from about 1000 microequivalents/g to about 8000 microequivalents/g. Although effective charge densities of more than about 8000 microequivalents/g can be used in the cleansing products of the present invention, such a large charge density is not typically required to realize the benefit of the present invention, and may result in the deterioration of product properties. As the effective charge density of the cationic material increases, the amount of cationic material required to be added to the pulp manufacturing process typically decreases. Generally, from about 0.01% (by weight of the substrate) to about 25% (by weight of the substrate), preferably from about 0.01% (by weight of the substrate) to about 10% (by weight of the substrate) of cationic material having the above-described effective charge density will be sufficient to increase the overall cationic charge of the resulting product sufficiently for purposes of the present invention. The actual amount of cationic material required for introduction into the pulp manufacturing process may be influenced by numerous other factors including, for example, the amount of steric hindrance in the pulp fibers due to other additives present in the pulp fiber environment, the accessibility of the charges on the pulp fibers, competitive reactions by cationic materials for anionic sites, the potential for multilayer adsorption into the pulp fiber, and the potential for precipitation of anionic materials out of solution.

Without being bound to a particular theory, it is believed that many of the cationic molecules (which may sometimes also be referred to as “softeners” or “debonders”) suitable for use in accordance with the present invention have a cationic charge by virtue of a quaternary nitrogen moiety. During the manufacturing of the skin cleansing product, this cationic charge may be used to attract the cationic molecule to the fiber surface, which is typically anionic in nature. The cationic compounds suitable for use in the present invention may have hydrophobic side chains which impart hydrophobicity to the molecule, making these molecules substantially non-water soluble. As such, these cationic compounds are believed to actually exist in solution as micelles of cationic compound molecules, where the hydrophobic tails are in the interior of the micelle and the cationic charges are exposed to the water phase. When a micelle cluster is adsorbed onto the fiber, more than one molecule is present on the surface, thus creating a site on the fiber with an excess of cationic charge. Once dried, these cationic molecules are likely associated with a counter-ion (although it may be possible that some are present without counter-ions which may create a static cationic charge) to form a net neutral charge. When the treated substrate comes into contact with an aqueous media such as the urine or feces, the counter-ion is free to dissociate and thus leaves the fiber cationically charged in the region with adsorbed cationic molecules. The cationic charge on the surface of the substrate is then able to attract and retain various microbes and/or contaminants which typically have a negatively charged outer surface.

### Section C

### Positive Charge Index Assay for Determining the Positive Charge Index of a Substrate

The amount of positive charge imparted onto a substrate, such as a base sheet or woven or non-woven web, for example, can be measured in accordance with the present invention using the Positive Charge Index Assay including an anionic dye binding assay. The Positive Charge Index Assay utilizes the dye Eosin Y, which is a biological stain for alkaline materials. Eosin B can optionally be utilized in place of Eosin Y. The Positive Charge Index Assay is carried out as follows:

Step 1: Cut the substrate to be evaluated into two squares approximately 2 centimeters by 2 centimeters. The first square will be stained with Eosin Y as described herein and optically evaluated. The second square will be subjected to the same Eosin Y staining procedure described herein with the exception that the second square will not be stained with Eosin Y; that is, the second square will undergo each and every step as the first square, except Steps 5 and 6 below.

Step 2: Introduce filter paper, such a Whatman #4 Qualitative 125 millimeter filter paper or equivalent, into a Buchner Funnel attached to a vacuum source.

Step 3: Start the vacuum, and wash the filter paper with deionized water.

Step 4: Allow the filter paper to dry.

Step 5: Place the test substrate on top of the dry filter paper and saturate the substrate with 0.75 milliliters of 0.5% (weight/volume) Eosin Y prepared in deionized water.

Step 6: Allow the test substrate to soak in the Eosin Y for 2 minutes and then cover the test substrate with a dry piece of filter paper.

Step 7: Wash the test substrate through the filter paper for 3 minutes with deionized water.

Step 8: Remove the test substrate with forceps and place it on a dry piece of filter paper and allow it to dry completely.

Step 9: Measure CIELAB Color Space of the dried test substrate using a Minolta CM-508d Spectrophotometer, or similar equipment. The spectrophotometer is set for CIELAB Color Space with the following parameters: Target Status CREEMM, Color Mode L*a*b*, Observer 10.degree., and the primary Illuminant D65. A standard white block supplied by the spectrophotometer manufacturer is utilized for calibration of the instrument.

Step 10: Calculate the DE*ab value of the Eosin Y stained test substrate using an un-stained test substrate for comparison. The DE*ab value is equal to the Positive Charge Index. The higher the Positive Charge Index, the higher the positive charge on the substrate. The CIE Color System Values are set forth below:

L*=Lightness=A value 0 to 100

a*=Color coordinate red-verses-green

b*=Color coordinate yellow-verses-blue

C=Chroma=[(a*).sup.2+(b*).sup.2].sup.1/2

h=Hue angle=arctan(b*/a*)

E=Color difference=[(L*).sup.2+(a*).sup.2+(b*).sup.2].sup.1/2

DL*=L*.sub.Eosin Stained Substrate-L*.sub.Unstained Substrate

Da*=a*.sub.Eosin Stained Substrate-a*.sub.Unstained Substrate

Db*=b*.sub.Eosin Stained Substrate-b*.sub.Unstained Substrate

DE*ab=[(DL*).sup.2+(Da*).sup.2+(Db*).sup.2].sup.1/2

The cationic compounds useful in the present invention to increase the overall effective cationic charge density of a finished product can easily be incorporated into various products. As used herein, the term “cationic compound” means any compound or ingredient which increases the overall cationic charge of the fibers comprising a cleansing product when the fibers are wetted. Preferably, the cationic compounds used in accordance with the present invention to increase the overall effective charge density of a finished product are non-antagonistic to pulp fibers or to other additives utilized in the manufacturing process. Further, it is preferred that the additional cationic compounds added to the pulp in accordance with the present invention do not substantially adversely affect the overall strength and integrity of the resulting modified product.

### Section D

### Empirical

### EXAMPLE

Antimicrobial Coating of Material

A Biodyne B membrane (0.45 μm pore size; 10 mm discs, Pall Corporation, East Hills, N.Y.) was coated with 100 μl of a 50% w/v urea hydrogen peroxide in water (Sigma Chemical St. Louis, Mo.). The coated membrane was allowed to dry over night at room temperature. The total add-on was 50 mg of urea peroxide per 78.5 mm² or 0.64 mg/mm².

Biodyne B Membrane Description

Pore surfaces populated by a high density of quaternary ammonium groups. This results in a positive surface charge over a broad pH range. Positive charge promotes strong ionic binding of negatively charged molecules.

Microbial Challenge Experiment

About 100 μl of a 6×10⁷ CFU/ml of Klebsiella pneumoniae ATCC 4352 suspension in phosphate buffered saline (pH 7.4) was added to the top of the Biodyne B membranes and allowed to incubate at 25° C. for 15 min. The exposed Biodyne B membranes were placed in 25 ml of Letheen broth and extracted by vortexing (20 sec) and shaking on an orbital shaker (10 min). Plating was done employing a spiral plater (WASP, Microbiological Associates) on trpyticase soy agar. Counts were done utilizing a digital imaging system (ProtoCOL, Microbiological Associates). A set of 5 replicates were done. Coated Biodyne B membranes were compared to uncoated Biodyne B membranes to determine Log₁₀ reductions.

TABLE 2
K. pneumoniae counts after 15 min exposure at 25° C. from
Biodyne B membrane samples that are urea peroxide treated and
untreated. All replicate values averages of triplicates.

CPU/Filter

| | Untreated Biodine B | Biodine B Membranes |
|---|---|---|
| Replicate | Membranes | Coated with Urea Peroxide |
| 1 | 2.1 × 10⁵ | 1.6 × 10³ |
| 2 | 3.0 × 10³ | 0.0E+00 |
| 3 | 5.4 × 10⁴ | 0.0E+00 |
| 4 | 2.6 × 10⁵ | 0.0E+00 |
| 5 | 2.4 × 10⁵ | 0.0E+00 |
| 6 | 5.2 × 10⁵ | 0.0E+00 |
| AVG | 2.7 × 10⁵ | 2.6 × 10² |

No significant or detectable population in replicates 2-4, biodine B charged membrane

Addition of 0.64 mg/mm² urea peroxide to a positively charge membrane provided at ≧3 Log₁₀ reduction of bacterial viability in 15 min at 25° C. It is expected that urea peroxide can added on to a positively charged modified substrate at concentrations ranging between 1-0.01 mg/mm² to produce adequate efficacy. Alternative peroxide types are: calcium peroxide, sodium carbonate peroxide, and carbohydrate peroxide mixtures that include dulcitol, arabitol, adonitol, mannitol, sorbitol, xylitol, lactitol, maltitol, dithioerythritol, dithiothreitol, glycerol, galactitol, erythritol, inositol, ribitol, and hydrogenated starch hydrolysates as the carbohydrate moiety. Types and add-on ranges of positively charged molecules would be expected to be in the range described in the following patent publications: U.S. 2004/0151919, U.S. 2004/0009141, U.S. 2004/0009210, and U.S. 2005/0137540, which are incorporated herein. This treatment type is applicable to woven, non-woven and/or formed polymers. Specific product forms are gloves, gowns, masks, drapes, wipes, diapers, air filters, and others.

The present invention has been described both in general and in detail by way of examples. Persons skilled in the art will understand that the invention is not limited necessarily to the specific embodiments disclosed. Modifications and variations may be made without departing from the scope of the invention as defined by the following claims or their equivalents, including equivalent components presently known, or to be developed, which may be used within the scope of the present invention. Hence, unless changes otherwise depart from the scope of the invention, the changes should be construed as being included herein.

We claim:

1. An oxidizing antimicrobial treatment comprising: preparing a substrate to accept an attachment of charged moieties, and a number of stabilized peroxide compounds on at least part of a surface of said substrate.

2. The treatment according to claim 1, further comprising associating microbes having an opposite charge of said charged moieties in close proximity to said substrate surface; activating and releasing peroxide molecules from said substrate surface; and killing said microbes.

3. The treatment according to claim 1, wherein said charged moieties are cationic that immobilize live bacteria on to said substrate surface.

4. The treatment according to claim 3, wherein said substrate comprises a sufficient amount of cationic compounds having an effective charge density of from about 0.1 microequivalents/g to about 8000 microequivalents/g or more.

5. The treatment according to claim 3, wherein said substrate surface is electrically altered to have a Positive Charge Index of at least about 35 positive charge units.

6. The treatment according to claim 1, wherein said peroxide compounds are associated directly on said substrate surface.

7. The treatment according to claim 1, wherein said peroxide compounds are within a number of degradable hollow structures, and are released upon contact with a source of moisture, or biological or microbial secretions.

8. The treatment according to claim 1, wherein said stabilized peroxide compounds is present on the substrate in an amount of up to about 20% by weight.

9. The treatment according to claim 1, wherein said peroxide release generates an oxidizing microenvironment when exposed to moisture associated with said bacteria.

10. The treatment according to claim 1, wherein said treated substrate generates a rate of at least 90% kill of microbes on a sample surface within 15 minutes of an initial contact.

11. The treatment according to claim 10, wherein said treated substrate kills about 95% of microbes on a sample surface within about 10 minutes of contact.

12. The treatment according to claim 1, wherein said peroxide molecules are contained in an agglomeration of particles.

13. The treatment according to claim 10, wherein said particles have a mean particle size of about 5 nm or smaller.

14. A protective article that minimizes contact transfer of microbes, said article comprises: a substrate surface, at least a portion of which is coated with a charged moiety and a number of stabilized peroxide compounds situated thereon, said stabilized peroxide molecules being adapted to release peroxide gas from said surface when reacted with a moisture-laden micro-atmosphere around said substrate.

15. The protective article according to claim 14, wherein said article has a body made in part from either a natural or synthetic latex film.

16. The protective article according to claim 15, wherein said latex firm is elastomeric.

17. The protective article according to claim 14, wherein said article has a body made in part from a non-woven substrate.

18. The protective article according to claim 14, wherein said article has a substrate made in part from a cellulose-based material.

19. The protective article according to claim 14, wherein said stabilized peroxide compounds is present on the substrate in an amount of up to about 20% by weight.

20. The protective article according to claim 19, wherein said stabilized peroxide compounds is present on the substrate in an amount of about 4-15% by weight.

21. The protective article according to claim 14, wherein part of an exterior or active surface of said substrate has a number of degradable hollow structures filed with peroxide agents.

22. The protective article according to claim 21, wherein said peroxide agents release from said hollow structures when said substrate contacts a source of significant moisture or specific biological or microbial secretions.

23. The protective article according to claim 22, wherein said hollow structures release said peroxide agents within in a prolonged, measured fashion onto said substrate surface.

24. The protective article according to claim 21, wherein said hollow structures release said peroxide agents within in a fast, explosive fashion onto said substrate surface.

25. The protective article according to claim 14, wherein said article is a glove.

26. The protective article according to claim 14, wherein said article is either a face mask, or a cover garment.

27. The article according to claim 14, wherein said protective article exhibit at least a 90% kill rate of microbes on a sample surface within about 15 minutes of contact.

28. The article according to claim 27, wherein said protective article exhibits a 95% or better microbe kill rate within about 10 minutes of contact.

29. An antimicrobial cleaning article comprising a at least a non-woven web body, having at least a first surface having a coating with an attachment of charged moieties, and a number of stabilized peroxide agents on at least part of a surface of said substrate.

30. The article according to claim 29, further comprises a cellulose-based material.

31. The article according to claim 29, wherein said cleaning article is a wiper sheet or pad.

32. The article according to claim 29, wherein said peroxide agents activate and release upon exposure to a sufficient amount of moisture associated with microbes.

33. The article according to claim 29, wherein said peroxide agent is stored in a degradable hollow structure associated as part of said substrate surface.

34. The article according to claim 29, wherein said cleaning article exhibits at least a 90% kill of microbes on a sample surface within about 15 minutes of contact.

35. The article according to claim 29, wherein said cleaning article kills about 90% of microbes on a sample surface within about 5 minutes of contact.

36. A process for treating a substrate with an oxidizing compound, the process comprises: providing a peroxide-containing compound, applying or incorporating said peroxide-containing compound to a surface of said substrate, wherein said peroxide-containing compound is generated in-situ in or on said substrate.

37. The process according to claim 36, wherein said peroxide-containing compound is generated according to either a freeze-dry method or a heated method, depending on the type or nature of said peroxide-containing compound and said substrate.
